(19) United States
(12) Patent Application Publication
(10) Pub. No.: US 2004/0197419 A1
(43) Pub. Date: Oct. 7, 2004

(54) Therapeutic methods, devices and compositions using combinations of naturally occurring elements

(76) Inventors: Theodore John Cole, Middletown, OH (US); Michael Christopher Peterson, West Chester, OH (US)

Correspondence Address: HASSE GUTTAG & NESBITT LLC, 7550 CENTRAL PARK BLVD., MASON, OH, 45040, US

(21) Appl. No.: 10/812,672

(22) Filed: Mar. 30, 2004

Related U.S. Application Data
(60) Provisional application No. 60/459,801, filed on Apr. 2, 2003.

(51) Int. Cl. A61K 33/24; A61K 33/38

(52) U.S. Cl. 424/617; 424/618

(57) ABSTRACT

Naturally occurring element combinations that are useful in methods, as well as compositions and devices, for carrying out vibrational therapies. These naturally occurring element combinations comprise: (a) at least one naturally occurring element selected from a first group consisting of charoite, pearl, rubellite, rebuilt with quartz, and ryolite; and (b) at least one naturally occurring element selected from a second group consisting of alexandrite, amazonite, amber, amethyst, aquamarine, aventurine, azurite, bloodstone, calcite, carelian, celestite, chrysoprase, citrine, copper, diamond, dioptase, emerald, fluorite, garnet, gold, hematite, Herkimer diamond, jade, jasper, kunzite pink, lapis lazuli, lepidolite, malchite, manganese, moonstone (e.g., rainbow moonstone), moss agate, obsidian, onyx (e.g., black onyx), opal, pearl, peridot, pyrite, quartz crystal, rhodochrosite, rhodonite, rose quartz, ruby, rutilated quartz, smoky quartz, sugilite, sodalite, sapphire, silver, tiger eye, topaz, tourmaline, turquoise and zircon.

### CROSS REFERENCE TO RELATED APPLICATIONS

This application claims the benefit of copending U.S. Provisional Patent Application Serial No. 60/459,801, filed Apr. 2, 2003.

### TECHNICAL FIELD

This invention relates to therapeutic methods, including methods for providing or improving the physical energy, well-being and support of individuals in need thereof, as well as providing pain relief, using combinations of naturally occurring elements selected from certain crystals, gem stones and/or metals. This invention further relates to therapeutic devices and compositions using such naturally occurring element combinations.

### BACKGROUND OF THE INVENTION

Many well-known non-invasive holistic therapeutic treatment methods operate by application of various items to the body of a person or animal. Pressure-oriented methods of holistic treatment include acupressure, which is used to stop pain and cure disease by applying pressure to key nerve centers about the body; massage techniques, which offer pain relief and muscle relaxation; and chiropractic medicine, which can even realign misaligned body parts. These techniques generally require training and practice to master, and are best utilized when a skilled practitioner applies the techniques to a patient, requiring expensive and time consuming personal attention.

In herbal medicine (phytopharmacognosy), treatments are often carried out through contact between the person to be treated and various plant materials. While some of these therapies involve breathing in odors or fumes produced from the herbs used, many simply require application of the herbs to the skin, in the form of lotions, poultices, tinctures, liniments, and the like. These treatments are known to provide pain relief, remedy skin irritations, relax tense muscles, and more. However, because herbal therapies rely on plant materials which eventually degenerate, they require that new materials be purchased periodically if the therapeutic technique is to be reapplied. Additionally, the herbs used in these therapies can create odors or skin sensations which are undesirable in some instances.

Other therapies have been developed which utilize naturally occurring materials including crystals, metals, and gemstones applied to the body to provide distinct therapeutic effects or benefits. These therapies, conventionally known as “crystal therapies,” are considered desirable because they use durable materials that can be used repetitively with no appreciable loss of function, produce no known detrimental side effects, and do not require professional supervision or application. It is thought that these crystals, metals, and gemstones enable what is called “vibrational support” or “vibrational therapies.” This belief is predicated upon the fact that crystals, such as quartz, vibrate at particular frequencies that play a role in placing a dysfunctional body back in harmonious synchronization. It is believed that the body vibrates at certain frequencies. In particular, molecules in the body, the greater part of which are water, continuously vibrate at certain frequencies. Under a healthy state or condition, these molecules vibrate at a certain frequency, whereas when the body is unhealthy, these molecules vibrate at another frequency. When any organ or part of the body is unable to maintain the ability to produce the vibrational equilibrium necessary to function at a healthy state, the energy of that organ or body part becomes deficient. This is known as a “misvibration.” Trauma to the human or animal body as a result of disease, injury, illness or natural depletion, for example, can also affect the healthy or desirable vibrations and thus cause a misvibration or a lower unhealthy vibration.

Crystal therapies are thought to aid in returning and/or keeping these molecules at vibrational frequencies that promote healthy conditions. Several techniques have used for treating and/or promoting the improved physical energy well being and support of an individual in need thereof, or in relieving pain, using such crystal therapies. For example, crystals have been hung or worn on the body. See U.S. Pat. No. 6,014,973 (Kaufman), issued Jan. 18, 2000 (method of healing comprising combination of five healing elements secured by flexible strand for wearing on body); U.S. Pat. No. 6,143,946 (Docter), issued Nov. 7, 2000 (therapeutic mat including relatively elastic substrate, hydrogel connected to substrate and plurality of microcrystals dispersed throughout hydrogel); U.S. Pat. No. 6,378,138 (Ridgley et al), issued Apr. 30, 2002 (stocking or hosiery that incorporates naturally occurring elements for healing various bodily ailments). Another technique employs crystals in an emollient cream that aids in retaining the crystals to the skin. See col. 1, lines 38-42 of U.S. Pat. No. 6,143,946 (Docter), issued Nov. 7, 2000. Other approaches have included the use of blends of various types of crystals for the treatment of certain functions of the body. See U.S. Pat. No. 6,014,973 (Kaufman), issued Jan. 18, 2000. Even so, the selection of suitable combinations of such naturally occurring elements to provide desired vibrational therapies is still relatively empirical.

### BRIEF DESCRIPTION OF THE INVENTION

This invention is broadly directed at naturally occurring elements, and in particular certain naturally occurring element combinations, that are useful in therapeutic methods, as well as compositions and devices, for carrying out vibrational support or vibrational therapies. These naturally occurring element combinations comprise:

(a) at least one naturally occurring element selected from a first group consisting of charoite, pearl, rubellite, rubellite with quartz, and ryolite; and

(b) at least one naturally occurring element selected from a second group consisting of alexandrite, amazonite, amber, amethyst, aquamarine, aventurine, azurite, bloodstone, calcite, carelian, celestite, chrysoprase, citrine, copper, diamond, dioptase, emerald, fluorite, garnet, gold, hematite, Herkimer diamond, jade, jasper, kunzite pink, lapis lazuli, lepidolite, malchite, manganese, moonstone, moss agate, obsidian, onyx, opal, pearl, peridot, pyrite, quartz crystal, rhodochrosite, rhodonite, rose quartz, ruby, rutilated quartz, smoky quartz, sugilite, sodalite, sapphire, silver, tiger eye, topaz, tourmaline, turquoise and zircon.

The naturally occurring element combinations of this invention, as well as the therapeutic methods, devices and compositions using such naturally occurring element combinations, provide a number of benefits. These benefits include providing or improving the physical energy, well-being and support of individuals desiring or needing same in both healthy and diseased states, as well as providing relief from various types of pains and aches such as muscle stiffness, headache, menstrual pain, sprain, inflammation, arthritis, carpal tunnel, bruises, broken bones and sports injuries.

### DETAILED DESCRIPTION OF THE INVENTION

As used herein, the term “comprising” means various components, steps and the like can be conjointly employed in this invention. Accordingly, the term “comprising” encompasses the more restrictive terms “consisting essentially of” and “consisting of.”

As used herein, the terms “crystal therapies,” “vibrational support” and “vibrational therapies” refer interchangeably to typically non-invasive treatments of the human body or other animal bodies, as well as areas or parts of such bodies, that rely on naturally occurring elements that are placed proximate to the body, an area or part of the body (e.g., an organ), or are inserted or ingested through orifices of the body (e.g. mouth or nose), to be treated, and that are believed to operate by altering or changing the vibrational frequency of the body, area or part of the body, or molecules within the body or area or part of the body being treated, from a dysfunctional, unhealthy, or less healthy state or condition, to a more harmonious or healthy state or condition.

As used herein, the term “naturally occurring element” generally refers to those naturally occurring elements such crystals, gem stones, metals and the like that allow for natural typically noninvasive treatment of the body and that are suitable for use in vibrational therapies. Naturally occurring elements useful in herein include, but are not limited to alexandrite, amazonite, amber, amethyst, aquamarine, aventurine, azurite, black onyx, bloodstone, calcite, carelian, celestite, charoite, chrysoprase, citrine, copper, diamond, dioptase, emerald, fluorite, garnet, gold, hematite, Herkimer diamond, jade, jasper, kunzite pink, lapis lazuli, lepidolite, malchite, manganese, moonstone, moss agate, obsidian, onyx, opal, pearl, peridot, pyrite, quartz crystal, rhodochrosite, rhodonite, rose quartz, rubellite, rubellite with quartz, ruby, rutilated quartz, ryolite, smoky quartz, sugilite, sodalite, sapphire, silver, tiger eye, topaz, tourmaline, turquoise, zircon, as well as various combinations of these elements.

It has been found that particular combinations of these naturally occurring elements can provide desirable therapeutic benefits in terms of improved or increased physical energy, well-being, support, pain relief, etc. These combinations comprise: (a) at least one naturally occurring element selected from a first group consisting of charoite, pearl, rubellite, rubellite with quartz, and ryolite; and (b) at least one naturally occurring element selected from a second group consisting of alexandrite, amazonite, amber, amethyst, aquamarine, aventurine, azurite, bloodstone, calcite, carelian, celestite, chrysoprase, citrine, copper, diamond, dioptase, emerald, fluorite, garnet, gold, hematite, Herkimer diamond, jade, jasper, kunzite pink, lapis lazuli, lepidolite, malchite, manganese, moonstone (e.g., rainbow moonstone), moss agate, obsidian, onyx (e.g., black onyx), opal, pearl, peridot, pyrite, quartz crystal, rhodochrosite, rhodonite, rose quartz, ruby, rutilated quartz, smoky quartz, sugilite, sodalite, sapphire, silver, tiger eye, topaz, tourmaline, turquoise and zircon. Particularly desirable naturally occurring elements from the first group include: (1) combinations of pearl and ryolite; and (2) combinations of charoite, rubellite, rubellite with quartz, pearl and ryolite. Particularly desirable naturally occurring elements from the second group include amethyst, aquamarine, black onyx, citrine, emerald, fluorite, garnet, moonstone, rainbow moonstone, moss agate, peridot, pyrite, quartz, ruby, sapphire, turquoise, tourmaline, sodalite, sugilite, as well as various combinations of these naturally occurring elements from this second group. The amount of each naturally occurring element selected from the first and second groups can vary, for example, in the range of from about 1 to about 10 parts, more typically from about 1 to about 2 parts. The naturally occurring element combinations can be in the form of homogeneous or substantially homogeneous mixtures or blends, or can simply involve combinations of these naturally occurring elements that are proximately associated with one another, as well as being proximate to the body, or area or part of the body being treated. Particularly suitable naturally occurring combinations for use herein include: (1) 1 part moss agate, 1 part amethyst, 1 part aquamarine, 1 part charoite, 1 part citrine, 1 part emerald, 1 part fluorite, 1 part garnet, 1 part peridot, 1 part rubellite, 1 part rubellite with quartz, 1 part ruby, 1 part sapphire, 1 part turquoise, 1 part pearl, 1 part pyrite, 1 part moonstone, 1 part ryolite, 1 part tourmaline and 1 part quartz; and (2) 1 part sugilite, 2 parts sodalite, 1 part black onyx, 1 part Herkimer diamond, 1 part pearl, 1 part pyrite, 1 part rainbow moonstone, 1 part ryolite and 1 part quartz.

The naturally occurring elements used in the combinations of this invention can be in their roughly natural states (as a rock, pellet, stone, ore, etc.). The naturally occurring elements can be in different forms, including particles, powders, cut stones, and the like. The naturally occurring elements can be shaped as desired into beads or other designs. Naturally occurring elements in the form of beads, whether spherical, prismatic, or otherwise, can be easily secured to a supporting article such as a bracelet, necklace, anklet or the like for wearing by the user.

Various devices can be used for securing the naturally occurring element combinations of this invention to the body, or the area or part of the body to be treated. One embodiment of such a securing device is a flexible strand or strap suitable for wearing upon the body. See, for example, the therapeutic mat of U.S. Pat. No. 6,143,946 (Docter), issued Nov. 7, 2000 (herein incorporated by reference). The strand or strap can be worn as a necklace, bracelet, anklet, etc., or can be wrapped around a particular site of pain or injury, such as a sprain or cut. The strap or strand can be provided with suitable mechanisms for securing the strap or strand to the body, or area or part of the body to be treated, such as, for example, Velcro fasteners, button or snap systems, and the like. See, for example, the therapeutic mat of U.S. Pat. No. 6,143,946 (Docter), issued Nov. 7, 2000 (herein incorporated by reference). An embodiment of a device for securing the naturally occurring elements to such a flexible strand is to form each naturally occurring element into the shape of a bead having a bore or hole or therethrough, with each hole being sufficiently sized to permit the flexible strap or strand to pass through it. By passing the flexible strand through the hole of each bead, the beads are thus secured to the flexible strand. Alternative embodiments for supporting the naturally occurring element combinations of this invention proximate to or in direct contact with the body include, but are not limited to, placing them within a pouch worn on or with the body, incorporating them into clothing such as hats, visors, gloves, shirts, pants, undergarments, hosiery, socks, coats, jackets, shoes and the like, jewelry such as bracelets, rings, earrings, necklaces, therapeutic products such as therapeutic or cosmetic body wraps, and the like. The naturally occurring element combinations of this invention can also be used in devices that provide other beneficial therapies, such as massage. See, for example, the therapeutic massaging apparatus disclosed in U.S. Pat. No. 6,007,501 (Cabados et al), issued Dec. 28, 1999 (herein incorporated by reference).

The naturally occurring element combinations of this invention can also be formulated as compositions for application, insertion or ingestion, for example by crushing, grinding, granulating or otherwise size reducing the naturally occurring elements into the form of a paste, powder, lotion, or cream that can be rubbed onto the body (e.g., as a body paint); by formulating the naturally occurring elements into compositions that can be sprayed from an aerosol container, by dispersing the naturally occurring elements in a food, beverage or in a source of water used in the home for drinking or bathing, or by placing the water, food, beverage or other solids or fluids to be used in vibrational therapy proximate to the naturally occurring element combinations. Compositions, for example, in the form of pastes, lotions or creams can be formulated to include a suitable dermatologically compatible fluid, semi-viscous, highly viscous or solid carrier such as eucerine, glycerin or VASELINE® for the ground naturally occurring element combinations. Typically, the naturally occurring element combinations are crushed or ground to form a powder that is then dispersed in a conductive liquid or gel media such as a solvent based adhesive comprising acetone.

If desired, the naturally occurring element combinations of this invention can be formulated as a composition such as a coating or paint for application either to the body (or area or part of the body), or to articles, such as clothing, jewelry, etc., made from various materials such as fabrics, wood, plastic, metal, leather, furs, etc., or components or materials used in making such articles, to be worn by, on or otherwise associated with the body. Such coatings or paints can be formulated for application to fabrics, fibers, yarns, threads, ropes, clothing, bedding (e.g., mattresses), furniture (e.g., chairs, tables, racks, seats, couches, beds, stands, desks, cabinets, furniture covers, etc.), bandages, surgical dressings, therapeutic wraps, sports wraps, hats, headbands, gloves, plaster body casts, jewelry or cloth patches of various sizes, shapes and types. Once applied to article, the composition can simply be allowed to dry so that is suitable for use. The naturally occurring element combinations of this invention can also be incorporated into the materials used to form the articles that are worn or otherwise associated with the user, such as plastic, leather or rubber products.

The naturally occurring element combinations of this invention, either alone, or more typically associated with one of various devices or compositions as previously described, can be positioned or placed proximate the body at or near the desired treatment site, which is typically the site of pain, inflammation, skin irritation, or injury. However, therapeutic effects or benefits can also be produced by supporting the naturally occurring element combinations of this invention anywhere proximate to the body, for example, by placing these element combinations on a supporting rack, stand, table, bed, chair or the like that the user is sitting on, using or near to. In carrying out vibrational therapies, the food, beverage or other liquids used can also be treated by placing them in proximity to the naturally occurring element combinations of this invention, as distinguished from the naturally occurring element combinations being dispersed in these compositions or device. The naturally occurring element combinations of this invention can be used to treat a variety of injuries, aches and pains and ailments including but not limited to, muscle stiffness, muscle stiffness, headache, menstrual pain, sprain, inflammation, arthritis, carpal tunnel, bruises, broken bones and sports injuries, as well as to improve the overall health, vitality, and energy of the user of these combinations.

As is often the case in other therapies, such as herbal therapy, it is not understood for each of these naturally occurring element combinations every characteristic of the elements that cause the treatment to occur. Wearing the naturally occurring element combinations of this invention proximate to or in direct contact with the body will typically provide the desired treatment benefit. Treatment effects or benefits can also be produced where the naturally occurring elements are placed near to the body rather than on the body. However, applications of the naturally occurring element combinations of this invention wherein the combinations are placed on the body are typically preferred.

Because naturally occurring elements used in the combinations of this invention are durable, there is typically no appreciable wear down through use. However, if it is determined that the energy stored in the naturally occurring elements has become depleted, the energy of these naturally occurring elements can be replenished, for example, by substituting new naturally occurring elements for original ones.

The amount of time for which the naturally occurring element combinations of this invention should be supported proximate to or in direct contact with the body in order to cause substantial treatment effects or benefits to occur will vary according to the individual. The naturally occurring element combinations are typically maintained in position proximate to the body, or the area or part of the body being treated, for at least about two minutes at a time, more typically at least about five minutes at a time. No undesirable side effects have been known to occur where the naturally occurring element combinations of this invention have been left proximate to the body, or area or part of the body being treated, for longer periods of time.

The naturally occurring element combinations of this invention, alone or in or with associated devices and compositions for using same, can also be provided with a set of instructions for how to use these the naturally occurring element combinations, optionally along with any devices and compositions associated with these combinations for use thereof. These instructions can be written or printed on the packaging that the naturally occurring element combinations of this invention are sold or distributed in or with, or on a sheet(s) of paper separately therefrom. Alternatively, the naturally occurring element combinations of this invention can be provided with computer software (e.g., in the form of a floppy disk(s), CD ROM disk(s) or other non-volatile electronic storage media) packaged therewith that provide the instructions on how to use these naturally occurring element combinations of this invention, or the instructions can be provided and obtained electronically (e.g., via the Internet) from a remote site, such as from a web site or computer server. The naturally occurring element combinations of this invention can be provided in the kit with the associated devices and compositions for employing or using same, can be provided as coatings or paints with instructions for how to apply the coating/paints to the body or to articles, such as clothing, to be worn on the body, or how to apply the coating/paints to the components or materials for making such articles.

While specific embodiments of this invention have been described, it will be apparent to those skilled in the art that various modifications thereto can be made without departing from the spirit and scope of this invention as defined in the appended claims.

What is claimed is:

1. A naturally occurring element combination, which comprises:
a. at least one naturally occurring element selected from a first group consisting of charoite, pearl, rubellite, rebuilt with quartz, and ryolite; and
b. at least one naturally occurring element selected from a second group consisting of alexandrite, amazonite, amber, amethyst, aquamarine, aventurine, azurite, bloodstone, calcite, carelian, celestite, chrysoprase, citrine, copper, diamond, dioptase, emerald, fluorite, garnet, gold, hematite, Herkimer diamond, jade, jasper, kunzite pink, lapis lazuli, lepidolite, malchite, manganese, moonstone, moss agate, obsidian, onyx, opal, pearl, peridot, pyrite, quartz crystal, rhodochrosite, rhodonite, rose quartz, ruby, rutilated quartz, smoky quartz, sugilite, sodalite, sapphire, silver, tiger eye, topaz, tourmaline, turquoise and zircon.

2. The combination of claim 1 wherein the first group of naturally occurring elements is selected from the group consisting of: (1) combinations of pearl and ryolite; and (2) combinations of charoite, rubellite, rubellite with quartz, pearl and ryolite.

3. The combination of claim 1 wherein the second group of naturally occurring elements is selected from the group consisting of amethyst, aquamarine, black onyx, citrine, emerald, fluorite, garnet, moonstone, rainbow moonstone, moss agate, peridot, pyrite, quartz, ruby, sapphire, turquoise, tourmaline, sodalite, sugilite, and combinations thereof.

4. The combinations of claim 1 wherein the amount of each naturally occurring element selected from the first and second groups is in the range from about 1 to about 10 parts.

5. The combinations of claim 4 wherein the amount of each naturally occurring element selected from the first and second groups is in the range from about 1 to about 2 parts.

6. The combination of claim 5 which is selected from the group consisting of: (1) combinations of moss agate, amethyst, aquamarine, charoite, citrine, emerald, fluorite, garnet, peridot, rubellite, rubellite with quartz, ruby, sapphire, turquoise, pearl, pyrite, moonstone, ryolite, tourmaline and quartz; and (2) combinations of sugilite, sodalite, black onyx, Herkimer diamond, pearl, pyrite, rainbow moonstone, ryolite and quartz.

7. A device capable of being worn on the body that has associated therewith the combination of claim 1.

8. The device of claim 7 which is a strap.

9. The device of claim 7 which is clothing.

10. Furniture having associated therewith the combination of claim 1.

11. A composition capable of being applied to the body or to an article used in connection with the body that comprises the combination of claim 1.

12. The composition of claim 11 that is a coating or paint.

13. A composition that is ingestible and which comprises the combination of claim 1.

14. The composition of claim 13 which is in the form of a beverage.

15. A composition which is in the form of a source of water for drinking or bathing which comprises the combination of claim 1.

16. A method of treating an individual in need of vibrational therapy which comprises the step of placing the combination of claim 1 proximate to the body of the user.

17. The method claim 16 wherein the combination is ingested.

18. The method of claim 16 wherein the combination is worn on the body of the user.

19. A kit including the combination of claim 1 and instructions for using the combination to treat an individual in need of vibrational therapy.

20. The kit of claim 19 which further includes a device or composition associated with the combination for use thereof in treating the individual.
